(19) United States
(12) Patent Application Publication
(10) Pub. No.: US 2008/0319528 A1
(43) Pub. Date: Dec. 25, 2008

(54) MODULAR ENDOPROSTHESIS WITH FLEXIBLE INTERCONNECTORS BETWEEN MODULES

(75) Inventors: Travis R. Yribarren, Coarsegold, CA (US); Randolf Von Oepen, Los Altos Hills, CA (US); Sanjay Shrivastava, Mountain View, CA (US); Richard Newhauser, Redwood City, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Abbott Park, IL (US)

(21) Appl. No.: 11/958,311

(22) Filed: Dec. 17, 2007

Related U.S. Application Data
(60) Provisional application No. 60/946,066, filed on Jun. 25, 2007.

(51) Int. Cl. A61F 2/82

(52) U.S. Cl. 623/1.15; 623/1.43

(57) ABSTRACT

A modular endoprosthesis is configured to have improved flexibility during and after deployment by having separate endoprosthetic modules that are interconnected by flexible interconnectors. The modular endoprosthesis includes a plurality of separate endoprosthetic modules positioned adjacently so that a first end of a first endoprosthetic module is adjacent to an end of a second endoprosthetic module and a second end of the first endoprosthetic module is adjacent to an end of a third endoprosthetic module. Additionally, the modular endoprosthesis includes a plurality of flexible interconnectors coupled to the plurality of separate endoprosthetic modules so as to interconnect the first end of the first endoprosthetic module with the end of the second endoprosthetic module with a first flexible interconnector, and interconnect the second end of the first endoprosthetic module with the end of the third endoprosthetic module with a second flexible interconnector.

### CROSS-REFERENCE TO RELATED APPLICATIONS

This U.S. patent application claims the benefit of U.S. provisional patent application Ser. No. 60/946,066, filed Jun. 25, 2007, with Travis R. Yribarren et al. as inventors, which provisional patent application is incorporated herein by specific reference in its entirety.

### BACKGROUND OF THE INVENTION

I. The Field of the Invention

The present invention is related to a modular endoprosthesis having interconnected modular components. More particularly, the present invention is related to a modular endoprosthesis having separate and independent endoprosthetic modules that are interconnected with flexible interconnectors so as to allow the independent endoprosthetic modules to move or flex with respect to each other.

II. The Related Technology

Stents, grafts, and a variety of other endoprostheses are well known and used in interventional procedures, such as for treating aneurysms, for lining or repairing vessel walls, for filtering or controlling fluid flow, and for expanding or scaffolding occluded or collapsed vessels. Such endoprostheses can be delivered and used in virtually any accessible body lumen of a human or animal, and can be deployed by any of a variety of recognized means. One recognized indication of an endoprosthesis, such as a stent, is for the treatment of atherosclerotic stenosis in blood vessels. For example, after a patient undergoes a percutaneous transluminal coronary angioplasty or similar interventional procedure, a stent is often deployed at the treatment site to improve the results of the medical procedure and reduce the likelihood of restenosis. The stent is configured to scaffold or support the treated blood vessel; if desired, it can also be loaded with a beneficial agent so as to act as a delivery platform to reduce restenosis or the like.

An endoprosthesis, such as a stent, is delivered by a catheter delivery system to a desired location or deployment site inside a body lumen of a vessel or other tubular organ. The intended deployment site may be difficult to access by a physician and often involves traversing the delivery system through a tortuous luminal pathway. Thus, it can be desirable to provide the endoprosthesis with a sufficient degree of flexibility during delivery to allow advancement through the anatomy to the deployment site. Moreover, it may be desirable for the endoprosthesis to retain structural integrity while flexing and bending during delivery.

A stent in a Superficial Femoral Artery (SFA) application can undergo axial, bending, torsional, and radial loading that can lead to cracks and fracture. The stent connection sections or connection elements that join the stent rings can also transmit stress from ring to ring under axial, bending, torsional, and radial loading. In addition, when the stent goes around a curve the connecting elements or sections require the portions of the ring apposed to the outside of the curve to lengthen and the portions of the ring apposed to the inside of the curve to shorten. Lengthening and shortening portions of the ring increases the maximum stress because the ring cannot expand evenly. This can result in crack formation and possible stent fracture. Fracture surfaces can have sharp edges that can cause injury to the patient.

Although various endoprostheses have been developed to address one or more of the aforementioned performance characteristics, there remains a need for a more versatile design that improves one or more performance characteristics without sacrificing the remaining characteristics.

Therefore, it would be advantageous to have an endoprosthesis configured to have improved flexibility during and after deployment. Also, it would be beneficial to have a modular endoprosthesis configured to allow for adjacent endoprosthetic modules to move or flex relative to each other to enhance delivery in tortuous luminal pathways. Additionally, it would be beneficial to have a modular endoprosthesis that allows for decoupling of the individual endoprosthetic modules so the individual endoprosthetic modules can move independently.

### BRIEF SUMMARY OF THE INVENTION

Generally, the present invention is related to a modular endoprosthesis that can be configured to have improved flexibility during and after deployment. Also, the modular endoprosthesis can be configured to allow for adjacent endoprosthetic modules to move or flex relative to each other to enhance delivery in tortuous luminal pathways. Additionally, the modular endoprosthesis can be configured to allow for decoupling of the individual endoprosthetic modules so the individual endoprosthetic modules can move independently.

In one embodiment, the present invention includes a modular endoprosthesis. The modular endoprosthesis includes a plurality of separate endoprosthetic modules positioned adjacently so that a first end of a first endoprosthetic module is adjacent to an end of a second endoprosthetic module and a second end of the first endoprosthetic module is adjacent to an end of a third endoprosthetic module and so on. Additionally, the modular endoprosthesis includes a plurality of flexible interconnectors coupled to the plurality of separate endoprosthetic modules so as to interconnect the first end of the first endoprosthetic module with the end of the second endoprosthetic module with a first flexible interconnector, and interconnect the second end of the first endoprosthetic module with the end of the third endoprosthetic module with a second flexible interconnector. With this configuration, the modular endoprosthesis is capable of bending, such as bending around a bend in a body lumen of a patient during deployment, by at least the first and second endoprosthetic modules being capable of moving with respect to each other by flexing, moving, or bending at the first flexible interconnector. Optionally, the endoprosthetic module includes at least one low stress zone that is coupled to at least one of the flexible interconnectors. It will also be appreciated that the flexible interconnectors may also provide additional independence of endoprosthetic modules in axial and torsional directions.

In one embodiment, the present invention includes a modular endoprosthesis for implanting within a curved vessel. The modular endoprosthesis includes the following: a plurality of separate endoprosthetic modules; and a plurality of flexible interconnectors coupled to and interconnecting the separate endoprosthetic modules, the plurality of flexible interconnectors limit axial movement of said plurality of separate endoprosthetic modules upon placement within the curved vessel.

In one embodiment, the present invention includes a modular endoprosthesis having the following: a plurality of separate endoprosthetic modules positioned longitudinally so that a first end of a first endoprosthetic module is oriented toward an end of a second endoprosthetic module and a second end of the first endoprosthetic module is oriented toward an end of a third endoprosthetic module; and a plurality of flexible interconnectors coupled to the plurality of separate endoprosthetic modules so as to interconnect the first end of the first endoprosthetic module with the end of the second endoprosthetic module with a first flexible interconnector and interconnect the second end of the first endoprosthetic module with the end of the third endoprosthetic module with a second flexible interconnector, wherein the first and second endoprosthetic modules are capable of moving with respect to each other as the first flexible interconnector flexes.

In one embodiment, the present invention includes a modular stent capable of bending when delivered through a bend in a body lumen of a patient. The modular stent includes first and second stent rings that are coupled together with an elongated flexible interconnector. As such, the first stent ring has a first end opposite of a second end, and the first end has a first opening that fluidly communicates with a second opening in the second end to define a first lumen. The second stent ring has a third end opposite of a fourth end, and the third end has a third opening that fluidly communicates with a fourth opening in the fourth end to define a second lumen. The elongated flexible interconnector has a flexible body defined by a first connector end opposite of a second connector end. The first connector end is coupled to the second end of the first stent ring and the second connector end is coupled to the third end of the second stent ring so that the first lumen is longitudinally aligned with the second lumen. As such, the modular endoprosthesis is capable of bending, such as bending around a bend in a body lumen of a patient during deployment, by at least the first and second endoprosthetic modules being capable of moving, flexing, or bending with respect to each other by bending at the elongated flexible interconnector.

In one embodiment, each of the flexible interconnectors includes a biocompatible material, such as a polymer. Also, the polymer can be biodegradable. Additionally, the polymer can contain an active agent, such as antithrombotics, anticoagulants, antiplatelet agents, thrombolytics, antiproliferatives, anti-inflammatories, agents that inhibit hyperplasia, inhibitors of smooth muscle proliferation, antibiotics, growth factor inhibitors, cell adhesion inhibitors, antineoplastics, antimitotics, antifibrins, antioxidants, agents that promote endothelial cell recovery, anti-allergic substances, radiopaque agents, and combinations thereof.

In one embodiment, the flexible interconnector is a cord, such as a suture. Additionally, at least one endoprosthetic module can include a channel that receives the cord. Further, the cord can include an anchor element that secures the cord to the endoprosthetic module. For example, the anchor element can be selected from the group consisting of a fastener, crimp, adhesive bead, clip, or swaged tube on the cord, or other structures that limit movement of an endoprosthetic module along the length of the flexible interconnector, and/or combinations thereof.

In one embodiment, the flexible interconnector is a graft material that is grafted between adjacent endoprosthetic modules.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a portion of an embodiment modular endoprosthesis having interconnected annular elements.

FIGS. 2A-2B illustrate an embodiment of a modular endoprosthesis having interconnected annular elements in a deployable orientation (FIG. 2A) and a deployed orientation (FIG. 2B).

FIGS. 3A-3C illustrate an embodiment of a modular endoprosthesis having interconnected annular elements in a deployable orientation (FIG. 3A-3B) and a deployed orientation (FIG. 3C).

FIG. 4 illustrates an endoprosthetic element having a channel for receiving a floating flexible interconnector.

FIG. 5 illustrates an endoprosthetic element having a channel for receiving a fixed flexible interconnector that is fixed in place by two anchor elements.

FIG. 6 illustrates an endoprosthetic element having a fixed flexible interconnector that is fixed in place by a knot.

FIG. 7 illustrates an endoprosthetic element having two channels for receiving a pair of partially fixed flexible interconnectors that are each fixed in place by a single anchor element.

FIGS. 8A-8B illustrate a pair of interconnected endoprosthetic elements that are interconnected by a flexible graft element.

### DETAILED DESCRIPTION

Generally, the present invention is related to a modular endoprosthesis that can be deployed in a target vessel, and maintain its structural integrity when subjected to a large range of loading conditions during day-to-day activity. In such vessels where an endoprosthesis is placed, activities can cause different loads to be placed on the endoprosthesis creating internal stresses in the endoprosthesis that can lead to material failures. However, the modular endoprosthesis can be configured to maintain structural integrity during axial, bending, radial, and torsion strains when the patient walks, sits, or performs any other activity. As such, the modular endoprosthesis of the present invention can retain structural integrity when subjected to various loads and stresses.

### I. Introduction

The present invention includes a modular endoprosthesis having separate endoprosthetic modules that are interconnected through a flexible interconnector element. In the instance of the endoprosthesis being a stent, the individual endoprosthetic modules or stent modules can be configured to supply sufficient radial force to treat the vessel, but do not communicate significant axial, bending, or torsion stresses to each other due to the flexible interconnector absorbing much of those stresses.

Flexing of the flexible interconnectors enable portions of the adjacently positioned endoprosthetic modules to move either toward or away from each other. This movement allows the endoprosthetic modules to go around the curves of a patient's tortuous anatomy during delivery. This movement also reduces loads, stresses or strain applied to the endoprosthesis and its endoprosthetic modules through movement of the vessel, into which the endoprosthesis is implanted, following implantation, (i.e., during walking, sitting, exercising, etc.) of an individual or animal into which the endoprosthesis was implanted. The interconnector flexing increases the spacing between adjacently positioned endoprosthetic modules apposed to the outside of the curve of a moved vessel, for instance, while decreasing the spacing between adjacently positioned endoprosthetic modules apposed to the inside of the vessel's curve. Optionally, the separate endoprosthetic modules can become substantially decoupled from each other after delivery.

In one embodiment, the present invention includes a modular stent that has stent rings or stent modules, which are substantially independent relative to each other, with adjacently positioned stent rings or stent modules being interconnected through a flexible interconnector so that each stent ring or stent module can move independently. The flexible interconnector element can optionally interconnect adjacent stent modules by being coupled to a low-stress area on each stent module. For example, the flexible interconnector can be a cord running through a channel within the structure of the stent module. The flexible interconnector element can interconnect adjacent stent modules by only extending from one stent module to the adjacent stent module, or a single flexible interconnector element can interconnect all of the stent modules by extending from a first terminal stent module to an opposite terminal stent module and through all of the intermediate stent modules.

The flexible interconnector element may also be made from a variety of materials and in a variety of forms, such as a suture. Accordingly, biocompatible sutures for use in surgical settings can be used or configured as the flexible interconnector element. For example, a biocompatible suture may be made from a polymer, such as a bioabsorbable polymer. The suture may be a monofilament or a multifilament, such as a braided construction. Optionally, the suture can be prepared from a biocompatible material that can serve the double-function as a drug delivery medium.

The channel containing the flexible interconnector can be located within each stent module at an area of low strain, such as the straight segment of the stent strut, or a feature created at a crown of the strut pattern. It is notable that these channels may have a variety of forms. For example the channel area may be circular, square, a hook, or the like. Also, the channels may be formed through the stent strut in the longitudinal, lateral, and/or the radial direction. The channels may be closed, or open, for example, in the form of a cleat.

Placement of the suture within the channels of each stent module can be accomplished by simply threading the suture through the channel. The suture may be secured within the channel by knots, fasteners, clips, adhesives, or other structures or techniques that can be coupled to the suture, channel, and/or other portion of the stent module to prevent stent module migration during or after deployment. Alternatively, the suture can be deformed at or proximate the channel by heat stamping, crimping, and the like at the appropriate location. Alternatively, a tubular member can be swaged upon the suture.

The interconnected stent modules can be substantially independent from each other so that axial, torsional, radial, or bending loads are not transmitted significantly between stent modules through the suture. The suture can provide for more accurate placement of the modular stent by limiting relative axial movement between the stent modules, as may be experienced during deployment around a bend in the vasculature.

An embodiment of a modular stent having a suture as the flexible interconnector that interconnects separate stent modules can include the following benefits: stent modules that are substantially independent of each other can reduce the risk of material failure due to variable loading conditions; relative axial movement between adjacent stent modules can be limited in order to reduce stent splay during deployment around a bend in the vasculature; suture materials are generally biocompatible; suture materials can be configured to be biodegradable; and the suture materials can act as a vehicle for the delivery of beneficial agents to the treatment site.

In one embodiment, the present invention includes a modular stent that has stent rings or stent modules, which are interconnected through a flexible interconnector element prepared from a flexible material grafted between the individual stent modules. Optionally, the flexible graft material interconnects adjacent stent modules by being coupled to a low-stress area on each stent module. The graft material can have higher elasticity and more flexibility than the stent material so that the modular stent preferentially moves, bends or flexes at the flexible graft material. Also, the graft material can be substantially more elastic and flexible to allow significant deflection under torsional, axial, and bending loads. By being elastic and flexible, the graft material can inhibit substantial transmission of loads or stresses between adjacent stent modules. Further, flexing of the flexible interconnectors enable portions of the adjacently positioned stent modules to move either toward or away from each other. This movement allows the stent to go around the curves of a patient's tortuous anatomy during deliver. This movement also reduces loads, stresses or strain applied to the stent through movement of the vessel, into which the stent is implanted, following implanting, i.e., during walking, sitting, exercising, etc. of an individual or animal into which the stent was implanted. The interconnector flexing increases the spacing between adjacently positioned stent modules apposed to the outside of the curve of a moved vessel, for instance, while decreasing the spacing between adjacently positioned stent modules apposed to the inside of the vessel's curve.

The graft material can interconnect adjacent stent modules by only extending from one stent module to the adjacent stent module, or a single graft material can interconnect all of the stent modules by extending from a first terminal stent module to an opposite terminal stent module and through all of the intermediate stent modules.

Additionally, the graft material can allow adjacent stent modules to flex or bend with respect to each other, while keeping the adjacent stent modules interconnected. When the modular stent is deployed around a bend, the graft material can provide additional structural form to minimize the splay between the adjacent stent modules. This can avoid the possibility of strut module migration during and/or after deployment, and can ensure accurate stent module placement, such as around a vessel bend. As such, the graft material allows the modular stent to be delivered as a unitary endoprosthetic, and allows the individual stent modules to move, bend or flex independently so that less or no loads or stresses are transferred from one end of the modular stent to the other.

The graft material may also be made from a variety of materials and in a variety of forms or configurations. The graft material can be prepared from an elastic and flexible biocompatible material, with such material having a tubular, planar, and/or elongate configuration. For example, a biocompatible graft material may be made from a polymer such as an elastomer or the like. Also, the graft material can be prepared from a bioabsorbable polymer, such as polyhydroxyalkanoate, polyester amide, poly-L-lactide-co-glycolide, poly-dL-lactide-co-glycolide, chitosan, PBT, 4-hydroxybutyrate, 3-hydroxybutyrate, PEG, or the like. A biodegradable graft material can degrade and be absorbed within the body, and the time for degradation can be complete only after delivery of the modular stent, thereby allowing complete decoupling of adjacent stent modules. Optionally, the graft material can be prepared from a biocompatible material that can serve the double-function as a drug delivery medium. As such, the graft material can act as drug carrier for drug, such as an anti-inflammatory drug or any other type of beneficial drug used in conjunction with endoprostheses.

An embodiment of a modular stent having a graft material as the flexible interconnector can include the following benefits: stent modules that are substantially independent of each other can reduce the risk of material failure due to variable loading conditions; relative axial movement between adjacent stent modules can be limited in order to reduce stent splay during deployment around a bend in the vasculature; the graft materials can be selected to be biocompatible; the graft materials can be configured to be biodegradable; the graft materials can act as a vehicle for the delivery of beneficial agents to the treatment site; the graft material can provide adequate structural form to ensure accurate placement of the modular stent and better vessel scaffolding; and in the case of a bioabsorbable graft material, complete decoupling of the stent modules can occur following graft degradation, which may also be timed to occur after delivery of the modular stent.

### II. Modular Endoprosthesis

In accordance with the present invention, a modular endoprosthesis can be provided for improved delivery within a body lumen of a human or other animal. Examples of modular endoprostheses can include stents, filters, grafts, valves, occlusive devices, trocars, aneurysm treatment devices, or the like. While the present invention is described in connection with stents, the principles can be applied to other types of endoprostheses.

A modular endoprosthesis can be configured for a variety of intralumenal applications, including vascular, coronary, biliary, esophageal, urological, gastrointestinal, or the like. The modular endoprosthesis can be prepared from multiple, separate annular elements or endoprosthetic modules that are interconnected by flexible interconnectors. As such, the interconnectors can inhibit loads, stresses, or strains from being transmitted between adjacent annular elements or endoprosthetic modules. The adjacent annular elements or endoprosthetic modules can be separated by flexible interconnectors that allow for isolation of loads, stresses, or strains within a particular annular element or endoprosthetic module. These flexible interconnectors enable portions of the adjacently positioned stent modules to move either toward or away from each other. This movement allows the stent to go around the curves of a patient's tortuous anatomy during delivery. This movement also reduces loads, stresses or strain applied to the stent through movement of the vessel, into which the stent is implanted, following implantation, (i.e., during walking, sitting, exercising, etc.) of an individual or animal into which the stent was implanted. The interconnector flexing increases the spacing between adjacently positioned stent modules apposed to the outside of the curve of a moved vessel, for instance, while decreasing the spacing between adjacently positioned stent modules apposed to the inside of the vessel's curve. In this manner, the overall structural integrity of the modular endoprosthesis can be improved over the life of the device. For example, the flexible interconnector can inhibit crack formation and propagation, and reduce the opportunity for the modular endoprosthesis to fail because loads, stress, or strain is reduced.

Generally, a modular endoprosthesis of the present invention can include a plurality of endoprosthetic modules each comprised of at least a first set of interconnected strut elements that cooperatively define the endoprosthetic module. A strut element can be more generally described as an endoprosthetic element or module element, wherein all well-known endoprosthetic elements can be referred to here as a “strut element” for simplicity. Each strut element can be defined by a cross-sectional profile as having a width and a thickness, and including a first end and a second end bounding a length. The strut element can be substantially linear, arced, rounded, squared, combinations thereof, or other configurations. The strut element can include a bumper, crossbar, link, linker, connector, interconnector, intersection, elbow, foot, ankle, toe, heel, medial segment, lateral segment, combinations thereof, or the like, as described in more detail below.

The endoprosthetic module can include a plurality of circumferentially-adjacent crossbars that are interconnected end-to-end by an elbow connection, intersection, or a foot extension. As such, an endoprosthetic module can include an elbow, intersection, or a foot extension (“foot”) extending between at least one pair of circumferentially-adjacent crossbars. The elbow or foot can thus define an apex between the pair of circumferentially-adjacent crossbars of the endoprosthetic module. Also, an intersection can have a shape similar to a cross so as to provide a junction between two coupled pairs of circumferentially-adjacent crossbars.

The elbow can be configured in any shape that connects adjacent ends of circumferentially-adjacent crossbars, and can be described as having a U-shape, V-shape, L-shape, or the like. An intersection can be configured in any shape that connects longitudinal and circumferentially adjacent crossbars, and can be described as having a cross shape, X-shape, H-shape, K-shape, or the like. The foot can have a foot shape having a first foot portion extending circumferentially from an end of one of the adjacent strut members and a second foot portion extending circumferentially from a corresponding end of the other of the circumferentially-adjacent strut members. In combination, the first and second foot portions generally define an ankle portion connected to a toe portion through a medial segment and the toe portion connected to a heel portion through a lateral segment.

As described herein, a modular endoprosthesis, in one configuration, can include two or more endoprosthetic modules. Each endoprosthetic module can generally define a ring-like structure extending circumferentially about a longitudinal or central axis. The cross-sectional profile of each endoprosthetic module can be at least arcuate, circular, helical, or spiral, although alternative cross-sectional profiles, such as oval, oblong, rectilinear or the like, can be used.

When the modular endoprosthesis includes multiple spaced apart endoprosthetic modules, a first endoprosthetic module is aligned longitudinally adjacent to a second endoprosthetic module along the longitudinal axis. The first and second endoprosthetic modules are interconnected by flexible interconnectors. As such, the interconnectors interconnect adjacent endoprosthetic modules so as to improve the structural integrity of the modular endoprosthesis by inhibiting the buildup or propagation of loads, stresses or strains at or through the interconnectors or by inhibiting propagation of loads, stresses or strains between adjacent endoprosthetic modules.

The endoprosthetic modules, alone or in combination, generally define a tubular structure (e.g., modular stent). For example, each endoprosthetic module can define a continuous closed ring such that the longitudinally-aligned endoprosthetic modules form a closed tubular structure (e.g., modular stent) having a central longitudinal axis.

Alternatively, each endoprosthetic module can define an open ring shape such that a rolled sheet, open tubular, or “C-shape” type structure is defined by the annular elements. That is, the endoprosthetic module is not required to be closed.

Furthermore, each endoprosthetic module can optionally define substantially a 360-degree turn of a helical pattern or spiral, such that the end of one endoprosthetic module can be joined through the flexible interconnector with the corresponding end of a longitudinally-adjacent annular element or endoprosthetic module to define a continuous helical pattern along the length of the modular endoprosthesis.

A. Interconnected Annular Elements

One configuration of the present invention includes a modular endoprosthesis configured to move, flex or bend during deployment and after being set. The moving, bending, or flexing increases the spacing between adjacently positioned endoprosthetic modules, such as annular elements, apposed to the outside of the curve of a moved vessel, for instance, while decreasing the spacing between adjacently positioned endoprosthetic modules apposed to the inside of the vessel's curve. By so doing, the movement reduces loads, stresses or strain applied to the endoprosthesis through movement of the vessel, into which the endoprosthesis is implanted, following implanting, i.e., during walking, sitting, exercising, etc. of an individual or animal into which the endoprosthesis was implanted.

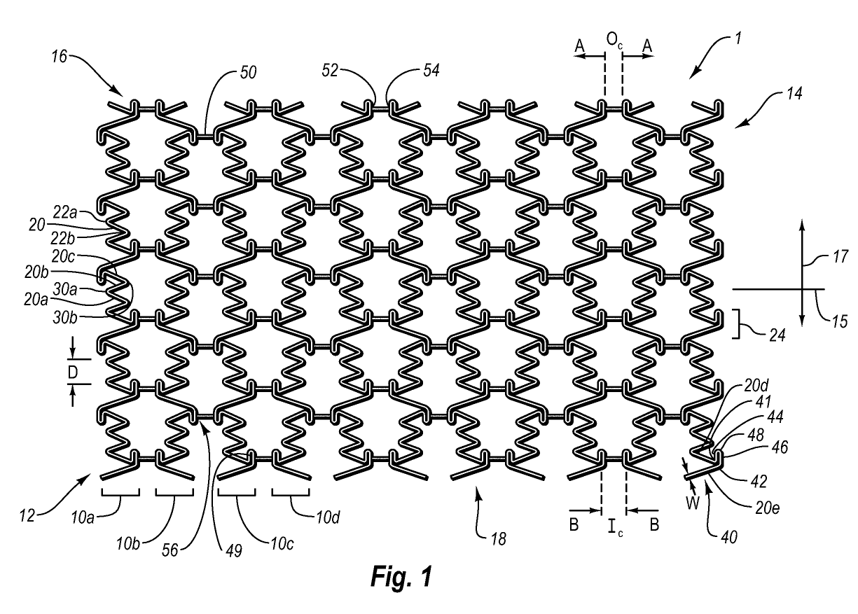

FIG. 1 illustrates an embodiment of a modular endoprosthesis that includes a plurality of annular elements (e.g., endoprosthetic modules) that are interconnected by a plurality of flexible interconnectors. The interconnectors function to reduce force transmission between adjacent annular elements, and thereby allow the individual annular elements to flex, move longitudinally, and/or bend with respect to each other while in a collapsed or deployed configuration. Additionally, the interconnectors allow the individual annular elements to flex, bend, or move radially, circumferentially, axially, and longitudinally while deployed.

FIG. 1 is a schematic representation of a side view of a portion of an embodiment of a modular endoprosthesis 1. The illustrated modular endoprosthesis 1 is a stent, but it will be understood that the benefits and features of the present invention are also applicable to other types of modular endoprostheses or other medical devices known to those skilled in the art. Further, although the following discussion is directed to one illustrative stent, it will be understood by those skilled in the art that various other stent configurations are possible and would benefit from the inclusion of one or more flexible interconnectors according to the present invention.

For purposes of clarity and not limitation, the modular endoprosthesis 1 is illustrated in a planar format. As shown, the modular endoprosthesis 1 includes a plurality of annular elements 10 aligned longitudinally adjacent to each other along a longitudinal axis 15. The annular elements 10 can also be referred to as stent rings because each element is usually in the form of a ring. Furthermore, the annular elements or stent rings can also be considered as endoprosthetic modules of a modular endoprosthesis. Although only two interconnected annular elements 10 need to be provided for the modular endoprosthesis 1, it is possible that an endoprosthesis includes a plurality of annular elements 10a-10d as shown in FIG. 1.

Each annular element 10 includes a set of interconnected strut elements, shown as strut crossbars 20, which are disposed circumferentially about the longitudinal axis 15; the circumferential direction is represented by arrow 17. Each crossbar 20 has a first end 22a and a second end 22b. The first end 22a of a selected crossbar 20a is interconnected to a second end 22b of a circumferentially-adjacent crossbar 20b at an elbow 30a at a first longitudinal side 12. Additionally, the circumferentially-adjacent crossbar 20b is interconnected to another circumferentially-adjacent crossbar 20c at an elbow 30b at a second longitudinal side 14. Accordingly, further circumferentially-adjacent crossbars 20 are interconnected through elbows 30 at opposing longitudinal sides 12, 14 of the annular element 10a.

Each annular element 10 can be expanded to a deployed configuration as shown in FIG. 1 by altering or opening the angle of the elbows 30 interconnecting the circumferentially-adjacent crossbars 20, or can be collapsed into a deployable configuration by closing the angle of the elbows 30. Also, circumferentially-adjacent elbows 30 on each longitudinal side 12, 14 of the annular element 10 are spaced apart by a circumferential distance D, such that each annular element 10 is expanded by increasing the distance D and collapsed by decreasing the distance D. At any given condition between the delivery configuration and the deployed configuration, the distance D can be balanced or constant from one set of circumferentially-adjacent elbows 30 to the next, or it can be varied if desired.

Selected elbows 30 on each longitudinal side 12, 14 of the annular element 10 can be defined by interconnecting corresponding ends 22a, 22b of circumferentially-adjacent crossbars 20a, 20b directly together to form a zigzag pattern of alternating U-shapes, V-shapes, L-shapes, combinations thereof, or the like when deployed. Alternatively, an elbow 30 can be provided between the corresponding ends 22a, 22b of adjacent crossbars 20a, 20b to form another contoured shape.

FIG. 1 also depicts an embodiment of a foot extension 40 that extends between a pair 24 of circumferentially-adjacent crossbars 20d, 20e of each annular element 10. As depicted, the foot extension 40 includes an ankle 41 that circumferentially couples an end 22 of one of the adjacent crossbars 20d to a medial segment 44. The medial segment 44 extends from the ankle 41 to a toe 48 that circumferentially couples the medial segment to a lateral segment 46. The lateral segment 46 extends from the toe 48 to a heel 42 that circumferentially couples the lateral segment to the next circumferentially-adjacent crossbar 20e. Accordingly, the juncture of the crossbar 20d and the medial segment 44 defines an ankle portion 41 of the foot extension 40; the juncture of the medial segment 44 and the lateral segment 46 defines a toe portion 48 of the foot extension 40; and the juncture of the lateral segment 46 and crossbar 20e defines heel portion 42 of the foot extension 40. Each portion of the foot extension 40, as well as each of the circumferentially-adjacent crossbars 20, can have a substantially uniform cross-sectional profile illustrated by a substantially uniform width W and thickness (not shown).

For purposes of discussion and not limitation, FIG. 1 shows that a toe portion 48 extends in a first circumferential direction a distance greater than the distance the heel portion 42 of the foot extension 40 extends in an opposite circumferential direction. As such, the entirety of the foot extension 40 extends in the circumferential direction of the toe portion 48. Furthermore, at least one of the medial segment 44 or lateral segment 46 can open foot region 49.

The adjacent annular elements 10a-10d are interconnected with an interconnector 50 having the form and flexibility for reducing force transmission between adjacent annular elements and allowing adjacent annular elements to move independently of each other. Stated another way, the interconnector 50 includes a flexible material that allows movement of adjacent annular elements 10a, 10b so that each annular element can function and be positioned independently of the other annular elements in the modular endoprosthesis 1. As such, the endoprosthesis 1 includes a plurality of interconnectors 50 to connect adjacent annular elements 10a, 10b or 10c, 10d. Each interconnector 50 allows the adjacent annular elements 10a, 10b or 10c, 10d or move or flex away from each other to allow adjacent annular elements to move or bend closer together. For instance, once implanted portions of the endoprosthesis 1 can move closer together or further away from each other during the activity of the patient receiving the implant. This movement can curve portions of the vessel with the implanted endoprosthesis 1. With this movement, the spacing O_{c} between adjacently positioned annular elements apposed to the outside of the curve of a moved vessel increases, and indicated by arrows A, while decreasing the spacing I_{c} between adjacently positioned annular elements apposed to the inside of the vessel's curve, as indicated by arrows B.

Accordingly, the interconnector 50 includes a first end 52 opposite of a second end 54. For example, in the illustrated configuration the first end 52 of the interconnector 50 is coupled to a foot extension 40 of an annular element 10a, and the second end 54 is coupled to a foot extension 40 of a longitudinally-adjacent annular element 10b. More particularly, the ends 52, 54 of each interconnector are coupled to a lateral segment 46 of each foot extension 40. Alternatively, the interconnectors 50 can be coupled to any portions of longitudinally-adjacent annular elements 10a, 10b. The interconnector couplings 56 are described in more detail below.

The modular endoprosthesis 1 can be easily deployed because of the improved flexibility provided within each annular element 10 or between longitudinally-adjacent annular elements 10a, 10b. As such, the flexible interconnectors 50 of longitudinally-adjacent annular elements 10a, 10b cooperate so as to enable the modular endoprosthesis 1 to bend around a tight corner in the vasculature. In part, this is because the interconnectors can bend, flex, or otherwise deform in shape so that while one side 16 of the adjacent annular elements 10a, 10b contracts, the second side 18 of the adjacent annular elements 10a, 10b expands. Also, the combination of elbows 30, foot extensions 40, and/or interconnectors 50 allow for radial, lateral, longitudinal, and cross forces to be isolated at one annular element 10a without being propagated to an adjacent annular element 10b. Such isolation of forces can inhibit crack formation in one annular element 10a and inhibit crack propagation between adjacent annular elements 10a, 10b. Moreover, the interconnectors 50 allow adjacent annular elements to move independently with respect to each other in radial, longitudinal, and cross directions.

B. Interconnected Stent Rings

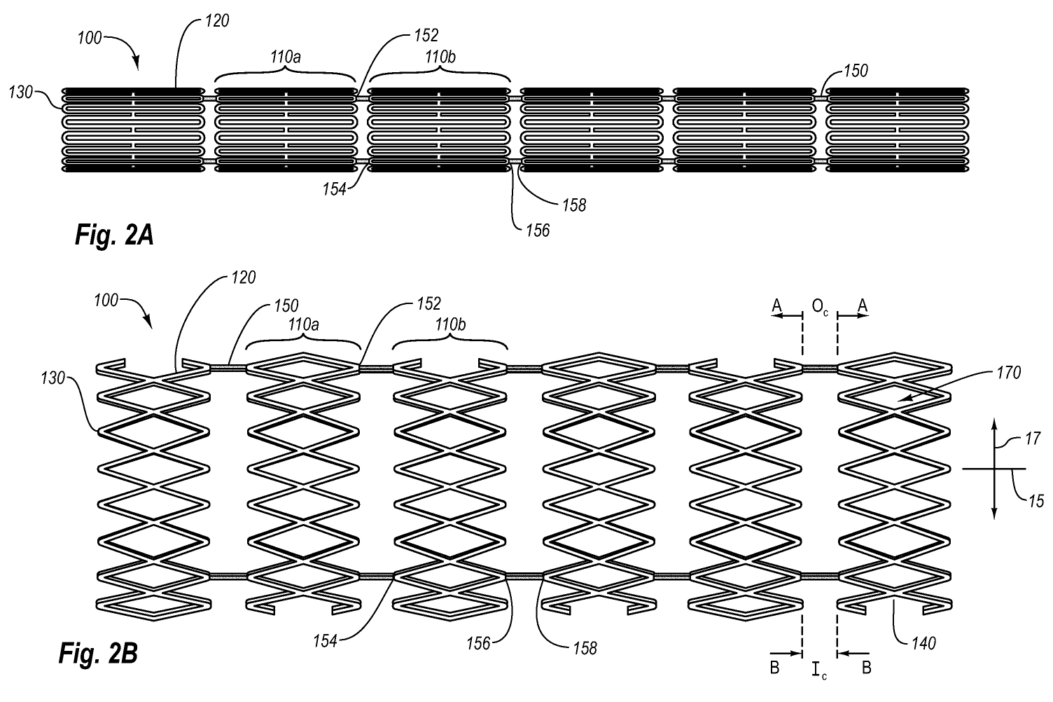

Another embodiment of the present invention includes a modular endoprosthesis having interconnected endoprosthetic modules that can move or flex with respect to each other during and after deployment. Accordingly, FIGS. 2A-2B illustrate another configuration of a modular endoprosthesis that can flex during deployment and separate into individual or interconnected annular elements after being deployed.

FIGS. 2A-2B provide side views of an embodiment of another modular endoprosthesis 100 in a collapsed delivery orientation (e.g., FIG. 2A) and an expanded deployed orientation (e.g., FIG. 2B). The discussions related to the modular endoprosthesis 1 of FIG. 1 can also apply to the modular endoprosthesis 100 of FIGS. 2A-2B. Accordingly, the modular endoprosthesis 100 can include a plurality of annular elements 110. The annular elements can be considered as stent rings or endoprosthetic modules of a modular endoprosthesis.

The plurality of annular elements 110 can have a plurality of crossbars 120 that are connected together by elbows 130 and intersections 140. More particularly, circumferentially-adjacent crossbars 120 can be coupled at an elbow 130 and four or more circumferentially-adjacent crossbars 120 can be coupled together at an intersection 140 as shown. However, other similar configurations for annular elements that are well known to be applied to endoprostheses can be utilized. With this configuration, crossbars 120, intersections 140, and elbows 130 cooperate so as to form a structure 170 that allows for flexibility as the modular endoprosthesis 100 or individual annular elements 110 can expand or collapse.

In the illustrated configuration, the structure 170 has a generally diamond shape that provides flexibility to each annular element 110 of the modular endoprosthesis 100. Thus, each annular element 110 has a series of circumferentially-interconnected flexible structures 170, such as, but not limited to, diamond structures, that can expand or collapse under the influence of a balloon or change of temperature. It will be understood that structure 170 can have other configurations or shapes while providing flexibility to the annular elements 110 of the modular endoprosthesis 100.

Additionally, the adjacent annular elements 110a, 110b are connected through a flexible interconnector 150. The interconnector 150 has a first end 152 coupled to a first annular element 10a and a second end 154 coupled to a second annular element 110b. Accordingly, the interconnector 150 includes a flexible material that allows movement of adjacent annular elements 110a, 110b so that each annular element can function and be positioned independently of the other annular elements in the modular endoprosthesis 100. As such, the modular endoprosthesis 100 includes a plurality of interconnectors 150 to connect adjacent annular elements 10a, 10b. Each interconnector 150 allows the adjacent annular elements 110a, 110b to move or flex away from each other to allow adjacent annular elements to move or flex closer together. For instance, once implanted portions of the endoprosthesis 100 can move closer together or further away from each other during the activity of the patient receiving the implant. This movement can curve portions of the vessel with the implanted endoprosthesis 100. With this movement, the spacing O_{c} between adjacently positioned annular elements apposed to the outside of the curve of a moved vessel increases, and indicated by arrows A, while decreasing the spacing I_{c} between adjacently positioned annular elements apposed to the inside of the vessel's curve, as indicated by arrows B.

FIG. 2A shows the modular endoprosthesis 100a in a collapsed orientation so that the annular elements 110a, 110b are contracted toward each other for deployment. Accordingly, the adjacent annular elements 110a, 110b are held together by the interconnector 150. In the contracted position, the interconnector 150 enables the annular elements 110a, 110b to flex, bend, or move with respect to each other in the radial, lateral, longitudinal, and cross directions. This allows the collapsed modular endoprosthesis 100a to flex and move without causing the annular elements 110 to expand or open. In part, this is because the couplings 156, 158 that connect the interconnector 150 to each annular element 110a, 110b can flex or move; the interconnector couplings 156, 158 are described in more detail below. Thus, each interconnector 150 can flex or move independently during deployment so that the annular elements 110a, 110b can move independently around tight corners without incurring undue stress.

FIG. 2B shows the modular endoprosthesis 100 in an expanded orientation so that the annular elements 110a, 110b extend away from each other. The adjacent annular elements 110a, 110b can be separated, but connected together by interconnectors 150 comprised of a flexible material or formed to be flexible. The configuration of the interconnectors 150 allows for the deployed annular elements 110a , 110b to flex with respect to each other in the radial, lateral, longitudinal, cross, and circumferential directions. In part, this is accomplished by the interconnector having ends 152, 154 with flexing couplings 156, 158, although other configurations of the members 150 can also achieve the desired functionality. The couplings 156, 158 and flexible interconnectors 150 allow the first annular element 110a to flex and/or move with respect to the second annular element 110b after being deployed so that each annular element functions as an independent endoprosthesis. Moreover, the flexible interconnectors 150 can cooperate with the elements or structures defining the structure 170 of the annular elements 110 so that the endoprosthesis 100 can flex, bend or move in any direction.

C. Interconnected Endoprosthetic Modules

Another embodiment of the present invention includes a modular endoprosthesis having interconnected endoprosthetic modules that can move with respect to each other during and after deployment. These endoprosthetic modules can be positioned adjacent to and in contact with each other when in a collapsed orientation and separate from each other while being interconnected when opened or expanded into a deployed orientation. The endoprosthetic modules can include bumpers that allow longitudinal forces to be transmitted throughout a portion or the entire modular endoprosthesis, thereby allowing the endoprosthetic modules to flex, move longitudinally, and/or bend with respect to each other while in a collapsed configuration. The bumpers of adjacent endoprosthetic modules can be connected together via an interconnection element as shown. Alternatively, the bumpers of adjacent endoprosthetic modules can be independent and not connected; however, the adjacent endoprosthesis can be interconnected via an interconnection element being linked to a member other than the bumper. In another alternative, adjacent endoprosthetic modules can be interconnected by having in interconnection element passing through a portion of each endoprosthetic module at any member thereof. Additional information regarding bumpers can be obtained in U.S. patent application Ser. No. 11/374,923, which is incorporated herein by specific reference.

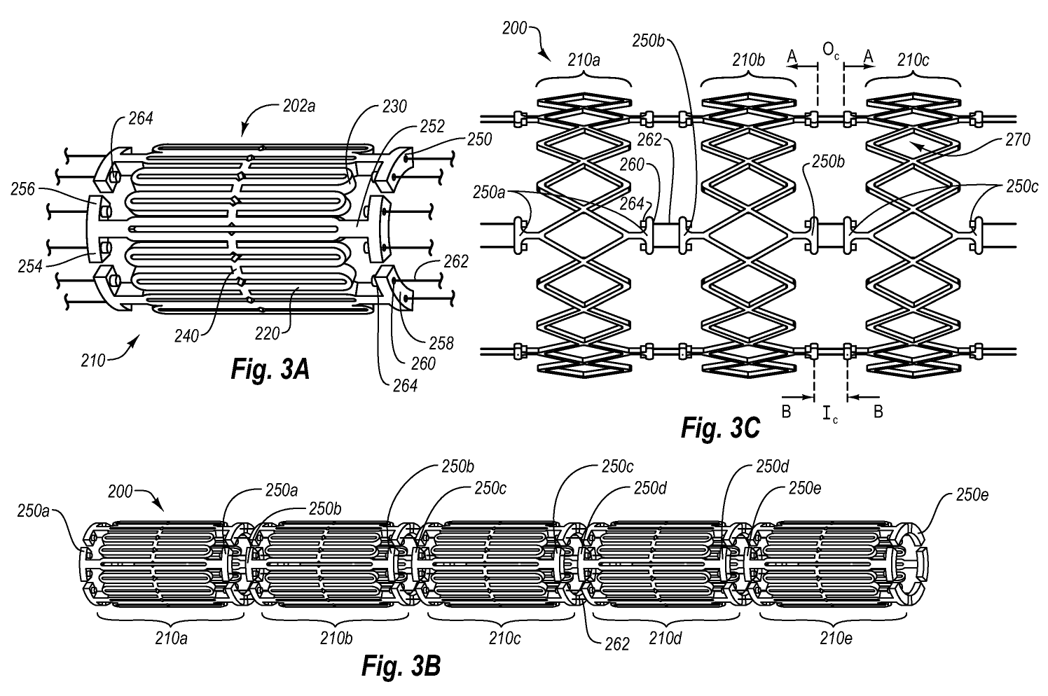

FIGS. 3A-3C illustrate another configuration of a modular endoprosthesis that can flex during deployment and separate into individual or interconnected endoprosthetic modules after being deployed. While the modular endoprosthesis shown in the figures have adjacent endoprosthetic modules being coupled via an interconnection element, the present invention could include coupled endoprosthetic modules being separated by a module that is not connected. For example, every other endoprosthetic module could be coupled together with an interconnection element, or every third endoprosthetic module could be similarly coupled together with an interconnection element. The endoprosthetic modules not directly coupled with their adjacent endoprosthetic module could be indirectly coupled with an interconnection element passing therethrough or thereabout or not coupled to the adjacent endoprosthetic module. Alternatively, a series of non-adjacent endoprosthetic modules elements can be coupled together via interconnection elements without or without the adjacent in endoprosthetic modules being indirectly coupled thereto or being coupled together. Examples of such interconnected endoprosthetic modules are described in more detail below.

FIGS. 3A-3C provide various views of a modular endoprosthesis 200 having independent endoprosthetic modules. As such, all elements described in connection with FIGS. 3A-3C are intended to be included in each of FIGS. 3A-3C. It will be understood that the structures, techniques, and teachings illustrated through FIGS. 3A-3B can also be applied to the structures of FIGS. 1-2B, and vice versa.

The modular endoprosthesis 200 (FIG. 3B) include a plurality of annular elements 210 (FIG. 3A) that each have a plurality of crossbars 220 that are connected together by elbows 230 and intersections 240. The intersections 240 that connect four crossbars 220 cooperate so as to form a structure 270 that allows for flexibility that can expand or collapse. Also, the annular elements 210 can be configured as described herein or as is well known in the art.

FIG. 3A shows an endoprosthetic module 210 in a collapsed orientation so that the crossbars 220 are collapsed toward each other so as to collapse each of the structures 270. More particularly, the elbows 230 and intersections 240 flex or bend so as to collapse each structure 270. Additionally, the endoprosthetic module 210 includes one or more bumpers 250 that include one or more ports 260 having an interconnector or interconnector element 262 extending therethrough. Each bumper 250 is coupled to an elbow 230 or other portion of the endoprosthetic module 210 through a neck 252 that longitudinally extends the bumper; however, other similar configurations can be used. The bumper 250 has a first arm 254 and a second arm 256 so as to form a T-shape with the neck 252. Also, the first arm 254 and second arm 256 are combined to form a bumper surface 258. However, the bumper 250 can have other shapes and configurations that can accommodate a port 260 for receiving an interconnector element 262.

As described, one or more of the arms 254, 256 of the bumper 250 includes a port 260 formed therein. The port 260 can be any type of hole that extends through the arm 254 so that the port 260 receives the interconnector element 262 extending therethrough. As shown, the interconnector element 262 includes an anchor element 264 to secure the interconnector element 262 to the endoprosthetic module 210. The anchor element 264 can be a clip, clasp, crimp, stopper, or other element that prevents the end of the end of the interconnector element 262 from slipping through the port 260.

FIG. 3B shows the modular endoprosthesis 200 in a collapsed orientation so that the endoprosthetic modules 210a-210e are contracted and held together for deployment. Accordingly, the adjacent endoprosthetic modules 210a-210e can be in contact through the bumpers 250a-250e. The bumpers 250a-250e allow the endoprosthetic modules 210a-210e to slide and separate from each other so that the endoprosthetic module can move relative to each other during and after deployment. However, the interconnector elements 262 keep adjacent endoprosthetic modules 210a-210b coupled together.

When the modular endoprosthesis 210a is in the contracted position, the bumpers 250 having the interconnector elements 262 enable the adjacent endoprosthetic modules 210a-210b to be held together and to move with respect to each other in longitudinal and cross directions. Also, this allows the collapsed modular endoprosthesis 200 to flex and bend without causing any of the endoprosthetic modules 210a-210e to expand or open. In part, this is because the bumpers 250 having the interconnector elements 262 allow the endoprosthetic modules 210 to move independently with respect to each other. Thus, each bumper 250 moves independently during deployment by the bumper surfaces 258 sliding with respect to each other or separating to the extent allowed by the interconnector element 262 so that the endoprosthetic modules 210a-210e move independently around tight corners without incurring undue stress.

FIG. 3C illustrates a portion of the modular endoprosthesis 200 of FIG. 3B in an expanded and deployed orientation. As such, the adjacent endoprosthetic modules 210a-210c are separated by the bumpers 250 having the interconnector elements 262. More particularly, the bumpers 250a of the first endoprosthetic module 210a separate from the bumpers 250b of the second endoprosthetic module 210b , but remain interconnected through the interconnector element 262. Additionally, the bumpers 250b of the second endoprosthetic module 210b separate from the bumpers 250c of the third endoprosthetic module 210c. In this configuration, the deployed endoprosthetic modules 210a-210c are capable of moving with respect to each other in the longitudinal, radial, cross, and circumferential directions. In essence, the modular endoprosthesis 200 is deployed into a plurality of separate and distinct endoprosthetic modules 210a-210c that are held together through a series of interconnector elements 262. Accordingly, the interconnector elements 262 allow movement of adjacent endoprosthetic modules 210a-210c so that each endoprosthetic module can function and be positioned independently of the other endoprosthetic module in the modular endoprosthesis 200. As such, each interconnector element 262 allows the adjacent endoprosthetic modules 210a-210c to move or flex away from each other to allow adjacent endoprosthetic modules 210a-210c to move or flex closer together. For instance, once implanted portions of the endoprosthesis 200 can move closer together or further away from each other during the activity of the patient receiving the implant. This movement can curve portions of the vessel with the implanted endoprosthesis 200. With this movement, the spacing O_{c} between adjacently positioned endoprosthetic modules apposed to the outside of the curve of a moved vessel increases, and indicated by arrows A, while decreasing the spacing I_{c} between adjacently positioned endoprosthetic modules apposed to the inside of the vessel's curve, as indicated by arrows B.

In one embodiment, the individual endoprosthetic modules described above, whether in FIG. 1, FIGS. 2A-2B, or 3A-3C, can be held together with a single interconnector element. This can include a single interconnector element being threaded through at least one port of each endoprosthetic module. As such, the independent endoprosthetic modules can slide over the interconnector and move with respect to each other, but stay interconnected through the interconnector. Also, a plurality of single interconnector elements can each be threaded through ports in all of the individual endoprosthetic modules of a modular endoprosthesis. Accordingly, the plurality of single interconnector elements can be located at different sides or portions of the individual endoprosthetic modules in order to simulate the tubular configuration of the modular endoprosthesis when the individual modules become separated.

In one embodiment, the modular endoprosthesis includes different types of interconnectors that are used to couple the endoprosthetic modules depending on the location of the modules and/or interconnectors with respect to each other and/or with respect to the shape or orientation of the body lumen. For instance, the modules adjacent to ends of the endoprosthesis, where axial stresses are high, having interconnectors that more resistant to axial motion, and modules located nearer to the middle of the endoprosthesis can be used in conjunction with an interconnector that resists torsional motion. Examples of such a configuration can include interconnectors described in connection to FIGS. 3A-3C to couple the modules in the middle of the endoprosthesis, and the interconnectors described in connection to FIGS. 2A-2B to couple the modules towards the ends of the endoprosthesis. Also, any variants of such combinations of different types of interconnectors can be employed.

### III. Endoprosthetic Composition

The endoprosthetic modules of the present invention can be made of a variety of materials, such as, but not limited to, those materials which are well known in the art of endoprosthesis manufacturing. This can include, but is not limited to, an endoprosthesis having a primary material for the annular elements, and a different material for the flexible interconnectors. Generally, the materials for the endoprosthetic modules can be selected according to the structural performance and biological characteristics that are desired. Materials well known in the art for preparing endoprostheses, such as polymers, ceramics, and metals, can be employed in preparing the endoprosthetic modules.

In one embodiment, the endoprosthetic modules can include a material made from any of a variety of known suitable materials, such as a shaped memory material (“SMM”). For example, the SMM can be shaped in a manner that allows for restriction to induce a substantially tubular, linear orientation while within a delivery shaft, but can automatically retain the memory shape of the endoprosthetic modules once extended from the delivery shaft. SMMs have a shape memory effect in which they can be made to remember a particular shape. Once a shape has been remembered, the SMM may be bent out of shape or deformed and then returned to its original shape by unloading from strain or heating. SMMs can be shape memory alloys (“SMA”) comprised of metal alloys, or shape memory plastics (“SMP”) comprised of polymers.

An SMA can have any non-characteristic initial shape that can then be configured into a memory shape by heating the SMA and conforming the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The main types of SMAs are as follows: copper-zinc-aluminium; copper-aluminium-nickel; nickel-titanium (“NiTi”) alloys known as nitinol. The nitinol alloys can be more expensive, but have superior mechanical characteristics in comparison with the copper-based SMAs, as well as better biocompatibility for medical applications. The temperatures at which the SMA changes its crystallographic structure are characteristic of the alloy, and can be tuned by varying the elemental ratios.

For example, the primary material of an endoprosthetic module can be of a NiTi alloy that forms superelastic nitinol. In the present case, nitinol materials can be trained to remember a certain shape, straightened in a shaft, catheter, or other tube, and then released from the catheter or tube to return to its trained shape. Also, additional materials can be added to the nitinol depending on the desired characteristic.

An SMP is a shape-shifting plastic that can be fashioned into an endoprosthetic module in accordance with the present invention. When an SMP encounters a temperature above the lowest melting point of the individual polymers, the blend makes a transition to a rubbery state. The elastic modulus can change more than two orders of magnitude across the transition temperature (“Tₜᵣ”). As such, an SMP can formed into a desired shape of an endoprosthetic module by heating it above the Tₜᵣ, fixing the SMP into the new shape, and cooling the material below Tₜᵣ. The SMP can then be arranged into a temporary shape by force, and then resume the memory shape once the force has been applied. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet determined. As such, any SMP can be used in accordance with the present invention.

For example, Veriflex™, the trademark for CRG's family of shape memory polymer resin systems, currently functions on thermal activation which can be customizable from −20° F. to 520° F., allowing for customization within the normal body temperature. This allows an endoprosthesis having at least one layer comprised of Veriflex™ to be inserted into a delivery catheter. Once unrestrained by the delivery shaft, the body temperature can cause the endoprosthetic module to return to its functional shape.

Also, it can be beneficial to include at least one layer of an SMA and at least one layer of an SMP to form a multilayered body; however, any appropriate combination of materials can be used to form a multilayered endoprosthesis.

Balloon-expandable endoprosthetic modules can be comprised of a variety of known suitable deformable materials, including stainless steel, silver, platinum, tantalum, palladium, cobalt-chromium alloys such as L605, MP35N, or MP20N, niobium, iridium, any equivalents thereof, alloys thereof, and combinations thereof. The alloy L605 is understood to be a trade name for an alloy available from UTI Corporation of Collegeville, Pa., including about 53% cobalt, 20% chromium and 10% nickel. The alloys MP35N and MP20N are understood to be trade names for alloys of cobalt, nickel, chromium and molybdenum available from Standard Press Steel Co., Jenkintown, Pa. More particularly, MP35N generally includes about 35% cobalt, 35% nickel, 20% chromium, and 10% molybdenum, and MP20N generally includes about 50% cobalt, 20% nickel, 20% chromium and 10% molybdenum.

Also, balloon-expandable endoprosthetic modules can include a suitable biocompatible polymer in addition to or in place of a suitable metal. The polymeric endoprosthetic module can include biodegradable or bioabsorbable materials, which can be either plastically deformable or capable of being set in the deployed configuration. If plastically deformable, the material can be selected to allow the endoprosthetic module to be expanded in a similar manner using an expandable member so as to have sufficient radial strength and scaffolding and also to minimize recoil once expanded. If the polymer is to be set in the deployed configuration, the expandable member can be provided with a heat source or infusion ports to provide the required catalyst to set or cure the polymer.

Additionally, a self-expanding configuration of an endoprosthetic module can include a biocompatible material capable of expansion upon exposure to the environment within the body lumen. Examples of such biocompatible materials can include a suitable hydrophilic polymer, biodegradable polymers, bioabsorbable polymers. Examples of such polymers can include poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like. For example, a self-expandable endoprosthetic module can be delivered to the desired location in an isolated state, and then exposed to the aqueous environment of the body lumen to facilitate expansion.

Furthermore, the endoprosthetic module can be formed from a ceramic material. In one aspect, the ceramic can be a biocompatible ceramic which optionally can be porous. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like. Optionally, the ceramic can be provided as sinterable particles that are sintered into the shape of an endoprosthetic module or layer thereof.

Moreover, the endoprosthetic module can include a radiopaque material to increase visibility during placement. Optionally, the radiopaque material can be a layer or coating any portion of the endoprosthesis. The radiopaque materials can be platinum, tungsten, silver, stainless steel, gold, tantalum, bismuth, barium sulfate, or a similar material.

### IV. Interconnectors

Generally, the modular endoprosthesis is comprised of endoprosthetic modules that are interconnected with a flexible interconnector element. The interconnector element can have various configurations in order to provide flexibility so that adjacent endoprosthetic elements can move independently while retaining interconnectivity. To provide the desired flexibility, each interconnector or interconnector element can (i) have a sufficient length and (ii) be capable of strain in at least one axis.

For example, the interconnectors can be prepared from cords that are coupled to each endoprosthetic module or a graft material that is deposited or otherwise attached to adjacent endoprosthetic modules. The cords can be any type of cord-like element having the size and characteristics sufficient for being tied to an endoprosthetic module or threaded through a port in the endoprosthetic module. The graft materials can be any type of material, such as a polymeric material, that can be applied to adjacent endoprosthetic modules in order to provide flexibility and mobility while retaining interconnectivity.

In one embodiment, the flexible interconnector element interconnects adjacent endoprosthetic modules by being coupled to a low-stress area on each endoprosthetic module. For example, the flexible material can be a suture material running through a channel within each endoprosthetic module or a flexible rail that is coupled to each endoprosthetic module. The flexible interconnector element can interconnect adjacent endoprosthetic modules by only extending from one endoprosthetic module to the adjacent endoprosthetic module, or a single flexible interconnector element can interconnect all of the endoprosthetic modules by extending from a first terminal endoprosthetic module to an opposite terminal endoprosthetic module and through all of the intermediate endoprosthetic modules.

A. Cord

One embodiment of the interconnector element can be a cord structure, such as a suture or the like. Accordingly, biocompatible sutures for use in surgical settings can be used or configured as the flexible interconnector element. This can include monofilament sutures, multifilament sutures such as braided sutures, or the like. For example, a biocompatible suture may be made from a polymer that is biostable or biodegradable. Optionally, the suture can be prepared from a biocompatible material that can serve the double-function as a drug delivery medium.

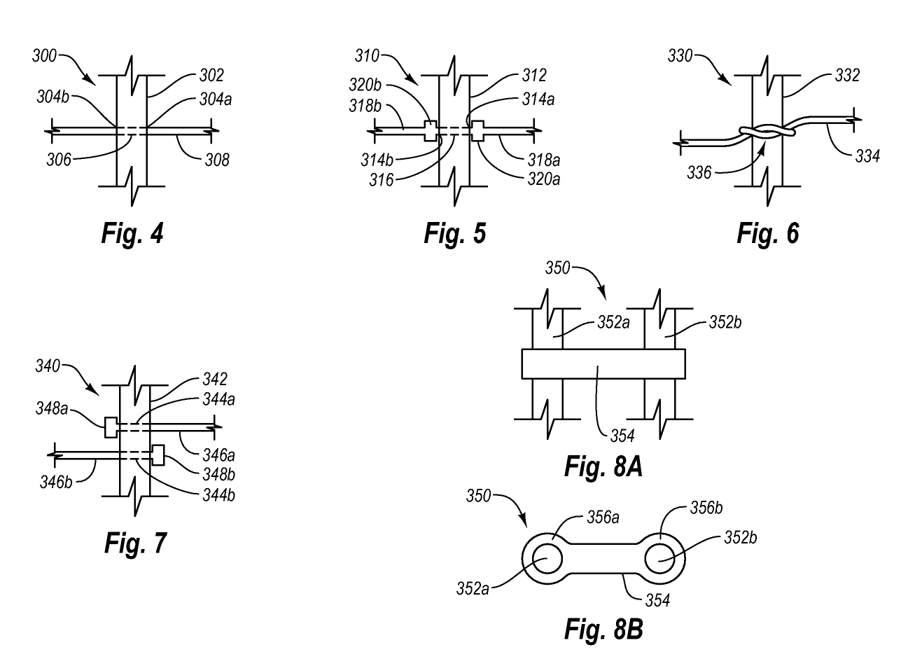

FIG. 4 is a side view of an interconnector system 300 that interconnects adjacent endoprosthetic modules (not shown) by being coupled to a module structure 302. The module structure 302 can be any portion of an endoprosthetic module as described herein or well known in the art. Only one module structure 302 is shown because the corresponding module structure of an adjacent endoprosthetic module can be substantially similar. As such, the module structure 302 is shown to include a first opening 304a fluidly coupled to a second opening 304b by a channel 306. The interconnector system 300 includes a cord 308 extending through the channel 306 so as to protrude from the first opening 304a and the second opening 304b. The cord 308 then extends to a single adjacent endoprosthetic module or multiple modules.

FIG. 5 is a side view of another interconnector system 310 that interconnects adjacent endoprosthetic modules (not shown) by being coupled to a module structure 312. As such, the module structure 312 is shown to include a first opening 314a fluidly coupled to a second opening 314b by a channel 316. The interconnector system 310 includes a cord 318 extending through the channel so as to protrude from the first opening 314a and the second opening 314b. The cord 318 includes an anchor element 320a, 320b on each side of the cord 318a, 318b. Each anchor element 320a, 320b can be a fastener, crimp, adhesive bead, clip, swaged tube, knot, or like element to prevent the channel 316 from sliding over either side of the cord 318a, 318b. The cord 318 then extends to a single adjacent endoprosthetic module or multiple modules.

FIG. 6 is a side view of another interconnector system 330 that interconnects adjacent endoprosthetic modules (not shown) by being coupled to a module structure 332. The interconnector system 330 includes a cord 334 that is tied around the module structure 332 with a tie structure 336. For example, the cord 334 can be tied around the module structure and secured with tie structure 336 being a knot. The cord 334 then extends to a single adjacent endoprosthetic module or multiple modules.

FIG. 7 is a side view of another interconnector system 340 that interconnects adjacent endoprosthetic modules (not shown) by being coupled to a module structure 342. As such, the module structure 342 is shown to include a first channel 344a and a second channel 344b. The interconnector system 340 includes a first cord 346a extending through the first channel 344a so as to protrude from the first channel on each side. The first cord 346a is secured to the module structure 342 by having a first anchor element 348a on one side of the first channel 344a so that the anchor element is inhibited from passing through the first channel. Additionally, the second cord 346b is secured to the module structure 342 by having a second anchor element 348b on one side of the second channel 344b so that the anchor element is inhibited from passing through the second channel. As shown, the first anchor 348a is disposed oppositely from the second anchor 348b; however, any orientation of multiple cords having multiple anchors can be used to prevent the cords from passing through their respective channels. Also, each cord 346 includes an anchor element 348 on each side of the channel 344.

Each channel can be located within a module structure of an endoprosthetic module at an area of low strain, such as the straight segment of a stent strut, or a feature created at a crown of the strut pattern. It is notable that these channels may have a variety of forms. For example the channel area may be circular, square, a hook, or the like. Also, the channels may be formed through the stent strut in the longitudinal, lateral, and/or the radial direction. The channels may be closed, or open, for example, in the form of a cleat.

Placement of the cord within the channels of each endoprosthetic module can be accomplished by simply threading the cord through the channel. The cord may be secured within the channel by an anchor element that can be coupled to the channel or other portion of the endoprosthetic module to prevent endoprosthetic module migration during or after deployment.

Additionally, while the FIGS. 4-7 show illustrations of cords extending in both directions from the module structures, such cords may extend in only one direction. As such, the cords can be terminally coupled to a module structure on one endoprosthetic module and only extend to the associated module structure on the adjacent endoprosthetic module. That is, a cord can have a first terminal end coupled with a first endoprosthetic module and a second terminal end coupled to the adjacent second endoprosthetic module. The terminal ends of the cord can be configured as described herein or well known in the art of tethering cords to structures. Accordingly, the features illustrated in the figures can be modified to similar features or utilize portions or combinations of features under the scope of the invention.

B. Graft

In one embodiment, the interconnector element can be prepared from a flexible material grafted between the individual endoprosthetic modules. The flexible graft material interconnects adjacent endoprosthetic modules by being coupled to a low-stress area on each endoprosthetic module. The graft material can have higher elasticity and more flexibility than the endoprosthetic material, and can be a polymer such as an elastomer or the like.

FIGS. 8A-8B show an interconnector system 350 that flexibly couples adjacent endoprosthetic modules (not shown). As such, FIG. 8A depicts a side view of the interconnector system 350 having a graft 354 coupled to a first module structure 352a of a first endoprosthetic module and to a second module structure 352b of a second endoprosthetic module. FIG. 8B is a cut-away top view that shows the graft 354 being coated around the first module structure 352a to form a first coupling 356a, and to a second module structure 352b to form a second coupling 356b.

While only one embodiment of an interconnector system employing a graft is depicted, other types of grafts and graft embodiments can be employed. For example, the module structure can be formed to interlock with the graft, or formed to include protrusions or recesses to receive the graft. Graft connectors can be arranged in a spiral manner, linear manner or in any other arrangement. Various graft materials can be employed to adjoin various modules within the same stent or device. Additionally, various techniques for depositing or coating graft materials can be employed to obtain a flexible graft that interconnects adjacent endoprosthetic modules.

Accordingly, the graft material can be substantially more elastic and flexible than the material of the endoprosthetic module to allow significant deflection under torsional, axial, and bending loads. By being elastic and flexible, the graft material can inhibit substantial transmission of loads between adjacent endoprosthetic modules. The graft material can interconnect adjacent endoprosthetic modules by only extending from one endoprosthetic module to the adjacent endoprosthetic module, or a single graft material can interconnect all of the endoprosthetic modules by extending from a first terminal endoprosthetic module to an opposite terminal endoprosthetic module and through all of the intermediate endoprosthetic modules.

Additionally, the graft material can allow adjacent endoprosthetic modules to flex or move independently, while keeping the adjacent endoprosthetic modules interconnected. When the modular endoprostheses is deployed around a bend, the graft material can provide additional structural form to minimize the splay between the adjacent endoprosthetic modules. This can avoid the possibility of endoprosthetic module migration during or after deployment, and can ensure accurate endoprosthetic module placement, such as around a vessel bend. As such, the graft material allows the modular endoprosthesis to be delivered as a unitary endoprosthesis, and allows the individual endoprosthetic modules to bend or flex independently so that less stress is transferred from one end of the modular endoprosthesis to the other.

C. Interconnector Materials

The interconnector elements of the present invention can be made of a variety of materials, such as, but not limited to, those materials which are well known in the art of biocompatible medical devices and sutures. Generally, the materials for the interconnector elements can be selected according to the structural performance and biological characteristics that are desired. Materials well known in the art for preparing biocompatible medical devices or sutures, such as polymers, ceramics, and metals, can be employed in preparing the interconnector elements.

The interconnector material can be prepared from an elastic and/or flexible biocompatible material that is biostable or biocompatible. The biostable or biocompatible material can be substantially similar to those described herein or well known in the art. For example, a biocompatible interconnector material may be made from a polymer, and preferentially from a bioabsorbable polymer, such as polyhydroxyalkanoate, polyester amide, poly-L-lactide-co-glycolide, poly-dL-lactide-co-glycolide, chitosan, PBT, 4-hydroxybutyrate, 3-hydroxybutyrate, PEG, or the like. A biodegradable interconnector material can degrade and be absorbed within the body, and the time for degradation can be complete only after delivery of the modular stent, thereby allowing complete decoupling of adjacent stent modules. Optionally, the interconnector material can be prepared from a biocompatible material that can serve the double-function as a drug delivery medium. As such, the interconnector material can act as drug carrier for drug, such as an anti-inflammatory drug or any other type of beneficial drug used in conjunction with endoprostheses.

In one configuration, the interconnector elements can be a biocompatible material. The biocompatible material can be biostable or biodegradable polymer. Examples of biostable polymers include polytetrafluorethylene (“PTFE”), expanded PTFE (“ePTFE”), Parylene®, Parylast® polyurethane (for example, segmented polyurethanes such as Biospan®), polyethylene, polyethylene terephthalate, ethylene vinyl acetate, silicone and polyethylene oxide. For example, the biodegradable polymer composition can include at least one of poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like.

Accordingly, the biodegradable material of the interconnector can contain a drug or beneficial agent to improve the use of the endoprosthesis. Such drugs or beneficial agents can include antithrombotics, anticoagulants, antiplatelet agents, thrombolytics, antiproliferatives, anti-inflammatories, agents that inhibit hyperplasia, inhibitors of smooth muscle proliferation, antibiotics, growth factor inhibitors, or cell adhesion inhibitors, as well as antineoplastics, antimitotics, antifibrins, antioxidants, agents that promote endothelial cell recovery, antiallergic substances, radiopaque agents, viral vectors having beneficial genes, genes, siRNA, antisense compounds, oligionucleotides, cell permeation enhancers, and combinations thereof.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A modular endoprosthesis for implanting within a curved vessel, comprising:
a plurality of separate endoprosthetic modules; and
a plurality of flexible interconnectors coupled to and interconnecting the separate endoprosthetic modules, the plurality of flexible interconnectors limit axial movement of said plurality of separate endoprosthetic modules upon placement within the curved vessel.

2. A modular endoprosthesis as in claim 1, wherein each of the flexible interconnectors includes a biocompatible material or a biodegradable material.

3. A modular endoprosthesis as in claim 1, wherein a combination of flexible interconnector each independently flex to move a first portion of each adjacently positioned endoprosthetic modules toward each other and an opposite second portion of each adjacently positioned endoprosthetic modules away from each other.

4. A modular endoprosthesis as in claim 1, wherein the flexible interconnector is a cord or a graft material.

5. A modular endoprosthesis as in claim 4, further comprising at least one anchor to anchor the cord to interconnect the interconnected endoprosthetic modules.

6. A modular endoprosthesis comprising:
a plurality of separate endoprosthetic modules positioned longitudinally so that a first end of a first endoprosthetic module is oriented toward an end of a second endoprosthetic module and a second end of the first endoprosthetic module is oriented toward an end of a third endoprosthetic module; and
a plurality of flexible interconnectors coupled to the plurality of separate endoprosthetic modules so as to interconnect the first end of the first endoprosthetic module with the end of the second endoprosthetic module with a first flexible interconnector and interconnect the second end of the first endoprosthetic module with the end of the third endoprosthetic module with a second flexible interconnector, wherein the first and second endoprosthetic modules are capable of moving with respect to each other as the first flexible interconnector flexes.

7. A modular endoprosthesis as in claim 6, wherein the flexible interconnector is a cord.

8. A modular endoprosthesis as in claim 7, wherein the cord is a suture.

9. A modular endoprosthesis as in claim 8, wherein at least one of the plurality of endoprosthetic modules includes a channel adapted to receive the cord.

10. A modular endoprosthesis as in claim 9, further comprising an anchor element that secures the cord to the at least one of the plurality of endoprosthetic modules.

11. A modular endoprosthesis as in claim 10, wherein the anchor element is selected from the group consisting of a fastener, crimp, adhesive bead, clip, swaged tube, and combinations thereof.

12. A modular endoprosthesis as in claim 6, wherein each endoprosthetic module of the plurality of endoprosthetic modules includes at least one low stress zone to which is coupled at least one of the plurality of flexible interconnectors.

13. A modular endoprosthesis as in claim 6, wherein modular endoprosthesis is a modular stent and the endoprosthetic modules are stent rings.

14. A modular endoprosthesis as in claim 6, wherein the flexible interconnector is a graft material that is grafted between adjacent endoprosthetic modules.

15. A modular endoprosthesis as in claim 14, wherein the graft material is loaded with a beneficial agent.

16. A modular endoprosthesis as in claim 15, wherein the beneficial agent comprises an antithrombotic, anticoagulant, antiplatelet agent, thrombolytic, antiproliferative, anti-inflammatory, agent that inhibits hyperplasia, inhibitor of smooth muscle proliferation, antibiotic, growth factor inhibitor, cell adhesion inhibitor, antineoplastic, antimitotic, antifibrin, antioxidant, agent that promotes endothelial cell recovery, antiallergic substance, radiopaque agent, viral vector having beneficial gene, gene, siRNA, antisense compound, oligionucleotide, cell permeation enhancer, or combinations thereof.

17. A modular endoprosthesis as in claim 6, where different types of interconnectors are used depending on the location of the coupled modules with respect to the endoprosthesis.

18. A modular endoprosthesis as in claim 17, wherein modules adjacent to ends of the endoprosthesis where axial stresses are high have interconnectors that are more resistant to axial motion, and modules located nearer to the middle of the endoprosthesis have interconnectors that are more resistant to torsional motion.

19. A modular stent capable of bending when delivered around a bend in a body lumen of a patient, the modular stent comprising:
a first stent ring having a first end and a first lumen extending from the first end;
a second stent ring having a second end and a second lumen extending from the second end toward the first end of the first stent ring; and
an elongated flexible interconnector having a flexible body defined by a first connector end opposite of a second connector end, the first connector end being coupled to the first end of the first stent ring and the second connector end being coupled to the second end of the second stent ring, the first and second endoprosthetic modules being capable of bending with respect to each other by bending at the elongated flexible interconnector.

20. A modular endoprosthesis as in claim 17, further comprising a third stent ring disposed between the first stent ring and the second stent ring, the third stent ring having a third lumen that receives the elongated flexible interconnector.

21. A modular endoprosthesis as in claim 17, further comprising a third stent ring having a third lumen and a fourth lumen formed in the second stent ring, the fourth lumen extending from the second side toward the first end of the first stent ring.

22. A modular endoprosthesis as in claim 19, further comprising a second elongated flexible interconnector having a flexible body, the second elongated flexible interconnector extending through the third lumen and the fourth lumen.
